(19) United States
(12) Patent Application Publication
(10) Pub. No.: US 2011/0297602 A1
(43) Pub. Date: Dec. 8, 2011

(54) LIQUID PURIFYING APPARATUS AND SUBSTRATE CLEANING APPARATUS

(75) Inventor: SHAO-KAI PEI, Tu-Cheng (TW)

(73) Assignee: HON HAI PRECISION INDUSTRY CO., LTD., Tu-Cheng (TW)

(21) Appl. No.: 12/825,356

(22) Filed: Jun. 29, 2010

(30) Foreign Application Priority Data
Jun. 4, 2010 (TW) 99118028

(51) Int. Cl. C02F 1/32

(52) U.S. Cl. 210/205

(57) ABSTRACT

An exemplary liquid purifying apparatus includes beads in contact with the liquid, a lighting element, and a driving member. Each bead has a purifying coating comprised of a nano material formed thereon. The lighting element has the beads positioned thereon. The driving member is configured for driving the lighting element to rotate, thereby rotating the beads in the liquid.

### CROSS-REFERENCE TO RELATED APPLICATION

This application is related to a commonly-assigned co-pending application entitled “liquid purifying apparatus” (Atty. Docket No. US33816). The disclosure of the above-identified application is incorporated herein by reference.

### BACKGROUND

1. Technical Field

The present disclosure relates to liquid purifying apparatuses, and particularly, to a liquid purifying apparatus having a lighting element and a substrate cleaning apparatus having same.

2. Description of Related Art

Liquids, such as tap water and recirculating water applications may have microorganisms which are poisonous and deleterious and need to be purified. Substrates such as glass substrates which easily have bacteria grown thereon, need to be cleaned in a liquid.

Various liquid purifying apparatuses in use are large complicated systems and involve long processes. It is known that during the long purifying process ambient contaminants around the apparatus may easily enter the liquid.

What is needed, therefore, is a liquid purifying apparatus and a substrate cleaning apparatus which can overcome the above shortcomings.

### BRIEF DESCRIPTION OF THE DRAWINGS

Many aspects of the present liquid purifying apparatus and substrate cleaning apparatus can be better understood with reference to the following drawings. The components in the drawings are not necessarily drawn to scale, the emphasis instead being placed upon clearly illustrating the principles of the present liquid purifying apparatus and substrate cleaning apparatus. Moreover, in the drawings, like reference numerals designate corresponding parts throughout the several views.

FIG. 1 is an isometric view of a liquid purifying apparatus in accordance with an exemplary embodiment.

FIG. 2 is a partially exploded view of the liquid purifying apparatus shown in FIG. 1.

### DETAILED DESCRIPTION

Embodiments of the present liquid purifying apparatus will now be described in detail below and with reference to the drawings.

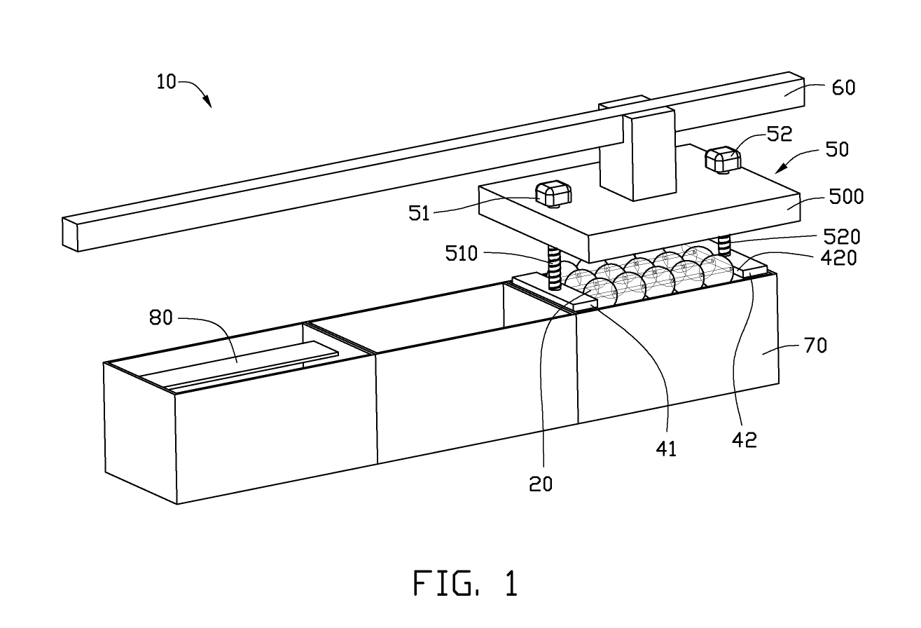

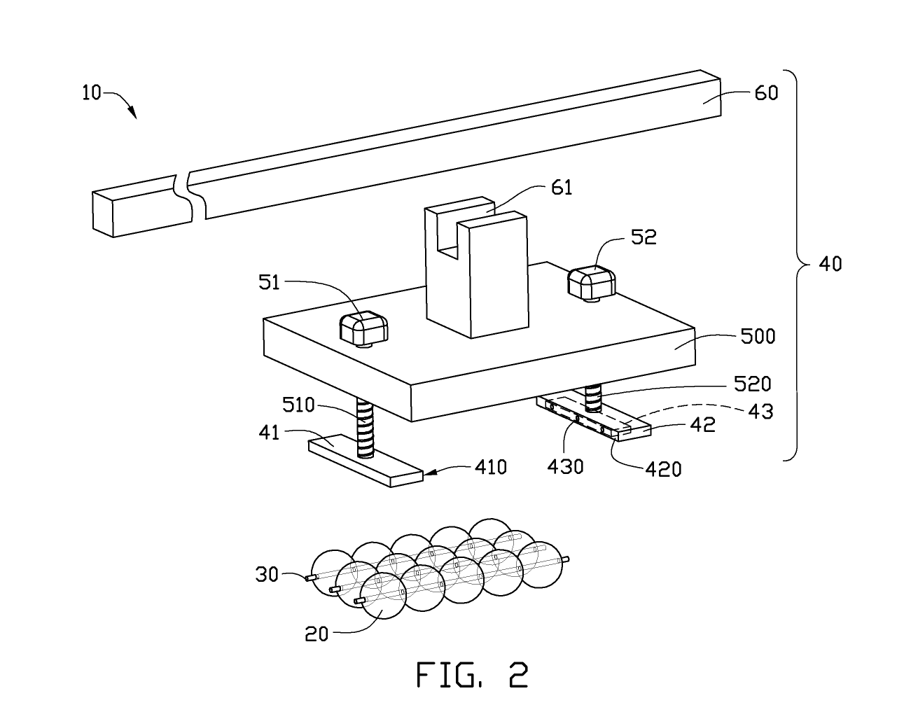

Referring to FIGS. 1 and 2, an exemplary liquid purifying apparatus 10 for purifying liquids such as tap water and recirculating water applications (e.g., recirculating aquarium water), is provided. The liquid purifying apparatus 10 includes beads 20, elongated lighting elements 30, a driving member 40, and a container 70.

Each bead 20 is made of a transparent glass, polymethyl methacrylate (PMMA) or ceramic material. The transparent ceramic material can be selected from a group consisting of aluminum oxide (Al₂O₃), magnesium oxide (MgO), beryllium oxide (BeO), yttrium oxide (Y₂O₃) and magnesium-aluminum oxide (MgAl₂O₄). Each bead 20 has a transparent purifying coating formed on the entire surface thereof. The beads 20 are substantially round, and a diameter of each bead 20 is in a range from 5 cm to 15 cm, and a thickness of the purifying coating is in a range from 55 nm to 100 nm. The beads 20 each can provide a large specific surface area to contact the liquid.

The transparent purifying coating is made of a nano titanium dioxide (TiO₂). The nano material has a high specific surface area. The TiO₂ has a good capability of removing impurities from the liquid, by decomposing certain organic materials into water and carbon dioxide (CO₂). Oxidation-reduction reaction may be enhanced by introducing the proper amounts of light to the process.

The lighting elements 30 are parallel with each other and each extends through a plurality of the beads 20. In this way, the beads 20 are positioned on the lighting elements 30 in contact with each other. In detail, the lighting elements 30 each can have a lighting diode and a transparent envelope enclosing the lighting diode therein. The lighting diode can be a white light diode or an ultraviolet (UV) light diode. The light comes from the lighting diode and transmits through the transparent materials of the beads 20. The TiO₂ can have a high activation for the purification under the light. The UV light can kill bacteria in the liquid.

The driving member 40 includes a guide rail 60, a lifting device 50, a first block 41, a second block 42, and a rotating motor 43. The rotating motor 43 is mounted on the second block 42. The first block 41 has a side surface 410 facing the second block 42, and the second block 42 has a side surface 420 facing the first block 41. The side surfaces 410, 420 each have a plurality of holes 430 formed therein. The lighting elements 30 extend through the holes 430 and are connected to the rotating motor 43. In this way, the lighting elements 30 together with the beads 20 can be driven to rotate by the rotating motor 43. The rotation can be about 180 degrees or about 360 degrees. The rotation ensures full involvement of all cleaning surfaces in the absorption, decomposition, and other reactions of the purifying process.

The container 70 is used to contain the liquid, and can include sections 71 for containing different liquids. The beads 20 are placed in contact with and rotated in the liquid to remove impurities from the liquid. The beads 20 and the container 70 can be configured so that the beads 20 cover most if not the entire surface of the liquid in the container 70, thus preventing ambient gas pollutants such as carbon monoxide (CO) and sulfur dioxide (SO₂) from entering the liquid.

The lifting device 50 includes a platform 500, an engaging block 61 formed on the platform 500, a first lifting motor 51 and a second lifting motor 52 both mounted on the platform 500. The lifting device 50 is slidably mounted on the guide rail 60 through the engaging block 61. The first lifting motor 51 has a first shaft connected to the first block 41, and the second lifting motor has a second shaft connected to the second block 42. The first lifting motor 51 and the second lifting motor 52 can help position the beads 20 in the liquid and remove the beads 20 from the liquid in the purification process.

In a situation, substrates 80 which have been polluted can be submerged in the container 70 which has a liquid such as tap water filled therein, then the liquid purifying apparatus 10 can work to remove microorganisms on the substrates, i.e., to clean the substrates.

It is understood that in several situations, only one lighting element is workable once the beads are positioned to contact and cover the surface of the liquid.

With the above liquid purifying apparatus 10, the liquid can be purified without large complicated systems and long processes.

It is understood that the above-described embodiments are intended to illustrate rather than limit the disclosure. Variations may be made to the embodiments and methods without departing from the spirit of the disclosure. Accordingly, it is appropriate that the appended claims be construed broadly and in a manner consistent with the scope of the disclosure.

What is claimed is:

1. A liquid purifying apparatus for purifying liquid, comprising:
a plurality of beads;
a purifying coating comprised of a nano material formed on each of the beads;
at least one lighting element with the beads positioned thereon; and
a driving member configured for driving the at least one lighting element to rotate, thereby rotating the bead in the liquid.

2. The liquid purifying apparatus of claim 1, wherein the at least one lighting element comprises a plurality of elongated lighting elements parallel with each other and each lighting element extending through a plurality of the beads.

3. The liquid purifying apparatus of claim 2, wherein the lighting element comprises a lighting diode and an elongated transparent envelope enclosing the lighting diode therein.

4. The liquid purifying apparatus of claim 2, wherein the beads are in contact with each other.

5. The liquid purifying apparatus of claim 2, wherein the beads are made of a transparent material.

6. The liquid purifying apparatus of claim 5, wherein the purifying coating is made of titanium dioxide.

7. The liquid purifying apparatus of claim 1, wherein the driving member comprises a first block and a second block with the beads arranged therebetween, and a rotating motor mounted in the first block and connected to the at least one lighting element.

8. The liquid purifying apparatus of claim 7, further comprising a lifting device having a platform, a first lifting motor and a second lifting motor both mounted on the platform, the first lifting motor having a first shaft connected to the first block, the second lifting motor having a second shaft connected to the second block.

9. The liquid purifying apparatus of claim 8, further comprising a guide rail for the platform sliding therealong.

10. The liquid purifying apparatus of claim 1, further comprising a container for containing the liquid.

11. The liquid purifying apparatus of claim 10, wherein the beads are arranged on the entire surface of the liquid in the container.

12. A liquid purifying apparatus comprising:
a plurality of transparent beads, a nano material transparent purifying coating formed on each of the beads;
a plurality of lighting elements extending through the beads for emitting light to the beads; and
a driving member configured for driving the lighting element to rotate, thereby rotating the beads.

13. The liquid purifying apparatus of claim 12, wherein a diameter of each bead is in a range from 5 cm to 15 cm, and a thickness of the purifying coating is in a range from 55 nm to 100 nm.

14. The liquid purifying apparatus of claim 12, wherein the purifying coating is made of titanium dioxide.

15. The liquid purifying apparatus of claim 12, wherein the driving member comprises a first block and a second block with the beads located therebetween, and a rotating motor mounted in the first block and connected to the lighting elements.

16. The liquid purifying apparatus of claim 15, further comprising a lifting device having a platform, a first lifting motor and a second lifting motor both mounted on the platform, the first lifting motor having a first shaft connected to the first block, the second lifting motor having a second shaft connected to the second block.

17. The liquid purifying apparatus of claim 16, further comprising a guide rail for the platform sliding therealong.

18. The liquid purifying apparatus of claim 12, further comprising a container for containing the liquid, and the beads are arranged on the entire surface of the liquid in the container.

19. A substrate cleaning apparatus comprising:
a container containing liquid therein;
a substrate submerged in the liquid;
a plurality of beads in contact with the liquid;
a transparent photocatalyst coating formed on each bead;
a plurality of lighting elements extending through the beads for activating the photocatalyst coating; and
a driving member configured for driving the lighting elements together with the beads to rotate.
